(19) United States
(12) Patent Application Publication
(10) Pub. No.: US 2002/0059682 A1
(43) Pub. Date: May 23, 2002

(54) Enzymatic dyeing agent

(76) Inventors: Horst Hoeffkes, Duesseldorf (DE); Astrid Kleen, Erkrath (DE); Andrea Saettler, Duesseldorf (DE)

Correspondence Address: HENKEL CORPORATION, 2500 RENAISSANCE BLVD, STE 200, GULPH MILLS, PA, 19406, US

(21) Appl. No.: 09/864,616

(22) Filed: May 24, 2001

Related U.S. Application Data
(63) Continuation of application No. PCT/EP00/12894, filed on Dec. 18, 2000.

(30) Foreign Application Priority Data
Dec. 24, 1999 (DE) DE 199 62 882.3

(51) Int. Cl. A61K 7/13

(52) U.S. Cl. 8/405

(57) ABSTRACT

Agents for dyeing keratin fibers are claimed which, in a cosmetically acceptable carrier, comprises at least one dye precursor and a phenol-oxidizing enzyme which can be obtained from Stachybotrys species.

The present invention relates to agents for dyeing keratin fibers which comprise at least one phenol-oxidizing enzyme which can be obtained from Stachybotrys species, to corresponding methods of dyeing keratin fibers, and the use of a phenol-oxidizing enzyme which can be obtained from Stachybotrys species for oxidative hair dyeing.

Human hair is nowadays treated in very diverse ways with hair cosmetic preparations. These include, for example, the cleansing of the hair with shampoo, the care and regeneration with rinses and cures, and the bleaching, dyeing and shaping of the hair using dyeing agents, tinting agents, waving agents and styling preparations. In this connection, agents for changing or shading the color of head hair play a prominent role.

For temporary colorations, use is usually made of dyeing agents or tinting agents which comprise “substantive dyes” as dyeing component. These are dye molecules which attach directly to the hair and do not require an oxidative process to form the color. These dyes include, for example, henna, which has been known since time immemorial for the dyeing of body and hair. These colorations are usually significantly more sensitive toward shampooing than the oxidative colorations, meaning that an often undesired shade shift or even a visible “bleaching” occurs very much more rapidly.

For permanent, intense colorations with corresponding fastness properties, oxidation dyeing agents are used. Such dyeing agents usually comprise oxidation dye precursors, so-called developer components and coupler components. Under the influence of oxidizing agents or of atmospheric oxygen, the developer components form the actual dyes with one another or with coupling with one or more coupler components. The oxidation dyeing agents are characterized by excellent, long-lasting coloring results. For natural-looking colorations, it is usually necessary to use a mixture of a relatively large number of oxidation dye precursors; in many cases, substantive dyes are also used for the tinting.

The oxidative development of the coloration can, in principle, take place with atmospheric oxygen. However, preference is given to using a chemical oxidizing agent. Suitable oxidizing agents are persulfates, chlorites and, in particular, hydrogen peroxide or addition products thereof with urea, melamine and sodium borate. An approximately 2-9% strength aqueous hydrogen peroxide solution is usually used. The keratin fibers, in particular if they have already been permanently waved or bleached, can be damaged by the action of such high oxidizing agent concentrations, and in rare cases, skin irritations may also arise as a result of these high concentrations.

An important solution approach to this problem starts from the reduction of the oxidizing agent concentration. For this reason, in the past dye precursors have, on the one hand, been sought which, because of their chemical structure, can be oxidized by even relatively small amounts of hydrogen peroxide or by atmospheric oxygen. On the other hand, the use of enzymes as biocatalysts has been proposed which are able to catalyze the desired oxidation process with very little or entirely without hydrogen peroxide only in the presence of atmospheric oxygen.

German laid-open specification DE-A-21 55 390 describes an enzyme-activated, oxidative hair-dyeing method in which small amounts of H₂O₂ are used in combination with a peroxidase. EP-A1-0 310 675 also discloses enzymatic hair-treatment agents which comprise at least one dielectron-reducing oxidase which utilizes oxygen as acceptor. EP-B1-0 548 620 describes enzymatic hair dyeing agents in which the oxidation of the dye precursors is catalyzed by the use of a peroxidase. Finally, EP-A2-0 795 313 describes enzymatic hair dyeing agents which comprise an oxygen-oxidoreductase/substrate system and a peroxidase and, as coupler components, mandatorily an m-phenylenediamine derivative. However, all of these dyeing agents are not entirely convincing with regard to the achievable coloring result (intensity, shade, shine, fastness properties).

The technology of the readily oxidizable dye precursors has, like the enzymatic color development described hitherto, the disadvantage that, compared with traditional methods, poorer results are achieved especially with regard to the intensity, the shine and the fastness properties of the colorations.

The object of the present invention was therefore to provide dyeing agents for keratin fibers which permit gentle fiber treatment and at the same time ensure an excellent coloring result.

The present invention firstly therefore provides agents for dyeing keratin fibers comprising, in a cosmetically acceptable carrier, at least one dye precursor and at least one phenol-oxidizing enzyme which can be obtained from Stachybotrys species.

According to the invention, keratin fibers are understood as meaning furs, wool, feathers and, in particular, human hair.

Preference is given according to the invention to phenol-oxidizing enzymes which can be obtained from Stachybotrys species, as described in international laid-open specifications WO-A2-99/49020 and WO-A2-99/49010. In particular, reference is explicitly made at this juncture to the disclosure with regard to the isolation and purification of these enzymes in WO-A2-99/49020. According to the invention, purified enzyme preparations are preferred. Examples of Stachybotrys species which can be used according to the invention are Stachybotrys parvispora, Stachybotrys chartarum, Stachybotrys kampalensis, Stachybotrys theobromae, Stachybotrys bisbyi, Stachybotrys cylindrospora, Stachybotrys dichroa, Stachybotrys oenanthes and Stachybotrys nilagerica. Also with regard to the derivatization and the use forms of these enzymes, reference is expressly made to the disclosure of the aforementioned international laid-open specifications.

The enzyme can be incorporated into the dyeing agent itself; however, it is preferably formulated separately from the dye precursors and only added to the dyeing agent directly prior to use.

The enzyme is preferably used in an amount of from 0.001-1% by weight, based on the amount of protein in the enzyme and the overall dyeing agent.

The present invention is not subject to any limitations with regard to the dye precursors used in the dyeing agents according to the invention. The dyeing agents according to the invention can comprise, as dye precursors,

oxidation dye precursors of the developer and/or coupler type, and

precursors of nature-analogous dyes, such as indole and indoline derivatives, and mixtures of representatives of these groups.

The developer components usually used are primary aromatic amines containing another free or substituted hydroxyl or amino group situated in the para or ortho position, diaminopyridine derivatives, heterocyclic hydrazones, 4-aminopyrazole derivatives and 2,4,5,6-tetraaminopyrimidine and derivatives thereof.

According to the invention, it may be preferred to use a p-phenylenediamine derivative or one of its physiologically compatible salts as developer component. Particular preference is given to p-phenylenediamine derivatives of the formula (I)

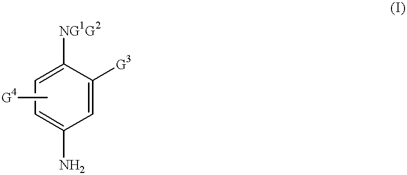

where

G¹ is a hydrogen atom, a C₁- to C₄-alkyl radical, a C₁- to C₄-monohydroxyalkyl radical, a C₂- to C₄-polyhydroxyalkyl radical, a (C₁- to C₄)-alkoxy-(C₁-to C₄)-alkyl radical, a 4′-aminophenyl radical or a C₁- to C₄-alkyl radical substituted by a nitrogen-containing group, a phenyl or a 4′-aminophenyl radical;

G² is a hydrogen atom, a C₁- to C₄-alkyl radical, a C₁- to C₄-monohydroxyalkyl radical, a C2- to C₄-polyhydroxyalkyl radical, a (C₁- to C₄)-alkoxy-(C₁-to C₄)-alkyl radical or a C₁- to C₄-alkyl radical substituted by a nitrogen-containing group;

G³ is a hydrogen atom, a halogen atom, such as a chlorine, bromine, iodine or fluorine atom, a C₁-to C₄-alkyl radical, a C₁- to C₄-monohydroxyalkyl radical, a C₁- to C₄-hydroxyalkoxy radical, a C₁-to C₄-acetylaminoalkoxy radical, a C₁- to C₄-mesylaminoalkoxy radical or a C₁- to C₄-carbamoylaminoalkoxy radical;

G⁴ is a hydrogen atom, a halogen atom or a C₁to C₄-alkyl radical or

if G³ and G⁴ are in the ortho position relative to one another, they can together form a bridging α,ω-alkylenedioxo group, such as, for example, an ethylenedioxy group.

Examples of C₁to C₄-alkyl radicals specified as substituents in the compounds according to the invention are the groups methyl, ethyl, propyl, isopropyl and butyl. Ethyl and methyl are preferred alkyl radicals. C₁- to C₄-alkoxy radicals preferred according to the invention are, for example, a methoxy or an ethoxy group. Furthermore, preferred examples of a C₁to C₄-hydroxyalkyl group which may be mentioned are a hydroxymethyl, a 2-hydroxyethyl, a 3-hydroxypropyl or a 4-hydroxybutyl group. A 2-hydroxyalkyl group is particularly preferred. Examples of halogen atoms are F, Cl or Br atoms according to the invention, Cl atoms being very particularly preferred. The other terms used are derived according to the invention from the definitions given here. Examples of nitrogen-containing groups of the formula (II) are, in particular, the amino groups, C₁to C₄-monoalkylamino groups, C₁to C₄-dialkylamino groups, C₁- to C₄-trialkylammonium groups, C₁to C₄-monohydroxyalkylamino groups, imidazolinium and ammonium.

Particularly preferred p-phenylenediamines of the formula (I) are chosen from p-phenylenediamine, p-tolylenediamine, 2-chloro-p-phenylenediamine, 2,3-dimethyl-p-phenylenediamine, 2,6-dimethyl-p-phenylene-diamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)-aniline, N,N-bis(β-hydroxyethyl)-p-phenylenediamine, 4-N,N-bis(β-hydroxyethyl)amino-2-methylaniline, 4-N,N-bis(β-hydroxyethyl)amino-2-chloraniline, 2-(β-hydroxyethyl)-p-phenylenediamine, 2-fluoro-p-phenylenediamine, 2-iso-propyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl,-P-hydroxyethyl)-p-phenylenediamine, N-(β,γ-dihydroxy-propyl)-p-phenylenediamine, N-(4′-aminophenyl)-p-phenylenediamine, N-phenyl-p-phenylenediamine, 2-(β-hydroxyethyloxy)-p-phenylenediamine, 2-(β-acetylaminoethyloxy)-p-phenylenediamine, N-(β-methoxyethyl)-p-phenylenediamine and 5,8-diaminobenzo-1,4-dioxane and their physiologically compatible salts.

Very particularly preferred p-phenylenediamine derivatives of the formula (I) according to the invention are p-phenylenediamine, p-tolylenediamine, 2-(β-hydroxyethyl)-p-phenylenediamine and N,N-bis(β-hydroxyethyl)-p-phenylenediamine.

It may also be preferred according to the invention to use compounds which contain at least two aromatic rings substituted by amino and/or hydroxyl groups as developer component.

Of the binuclear developer components which can be used in the dyeing compositions according to the invention, mention may be made in particular of the compounds which correspond to the following formula (II), and their physiologically compatible salts:

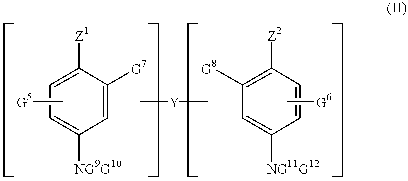

where

Z¹ and Z , independently of one another, are a hydroxyl or NH₂ radical which

is optionally substituted by a C₁- to C₄-alkyl radical, by a C₁- to C₄-hydroxyalkyl radical and/or by a bridge Y,

the bridge Y is an alkylene group having 1 to 14 carbon atoms,

such as, for example, a linear or branched alkylene chain or an alkylene ring which can be interrupted or ended by one or more nitrogen-containing groups and/or one or more hetero atoms such as oxygen, sulfur or nitrogen atoms, and may possibly be substituted by one or more hydroxyl or C₁- to C₅-alkoxy radicals,

G⁵ and G⁶, independently of one another, are a hydrogen or halogen atom,

a C₁- to C₄-alkyl radical, a C₁- to C₄-mono-hydroxyalkyl radical, a C₂- to C₄-polyhydroxyalkyl radical, a C₁- to C₄-aminoalkyl radical or a direct bond to the bridge Y,

G⁷, G⁸, G⁹, G¹⁰, G¹¹ and G¹², independently of one another are a hydrogen atom, a direct bond to the bridge Y or a C₁- to C₄-alkyl radical, with the proviso that the compounds of the formula (II) contain only one bridge Y per molecule.

The substituents used in formula (II) are defined according to the invention analogously to the above statements.

Preferred binuclear developer components of the formula (II) are, in particular: N,N′-bis(β-hydroxyethyl)-N,N′-bis(β-aminophenyl)-1,3-diaminopropanol, N,N′-bis(p-hydroxyethyl)-N,N′-bis(4-aminophenyl)ethylenediamine, N,N′-bis(4′-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino,3′-methylphenyl)ethylenediamine, 1,8-bis(2,5-diaminophenoxy)-3,5-dioxaoctane, bis(2-hydroxy-5-aminophenyl)methane, 1,4-bis(4-aminophenyl)diazacycloheptane and 1,10-bis(2,5-diaminophenyl)-1,4,7,10-tetraoxadecane and their physiologically compatible salts.

Very particularly preferred binuclear developer components of the formula (II) are N,N′-bis(β-hydroxyethyl)-N,N′-bis(4′-aminophenyl)-1,3-diaminopropanol, bis(2-hydroxy-5-aminophenyl)methane, N,N′-bis(4-aminophenyl)-1,4-diazacycloheptane and 1,10-bis(2,5-diaminophenyl)-1,4,7,10-tetraoxadecane or one of their physiologically compatible salts.

Furthermore, it may be advantageous according to the invention to use a p-aminophenol derivative or one of its physiologically compatible salts as developer component. Particular preference is given to p-aminophenol derivatives of the formula (III)

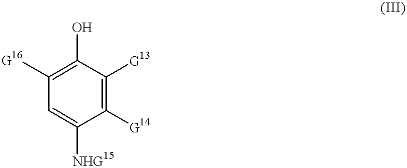

where:

G¹³ is a hydrogen atom, a halogen atom, a C₁- to C₄-alkyl radical, a C₁to C₄-monohydroxyalkyl radical, a (C₁- to C₄)-alkoxy-(C₁- to C₄)-alkyl radical, a C₁- to C₄-aminoalkyl radical, a hydroxy-(C₁- to C₄)-alkylamino radical, a C₁- to C₄-hydroxyalkoxy radical, a C₁- to C₄-hydroxyalkyl-(C₁- to C₄)-aminoalkyl radical or a (di-C₁to C₄-alkylamino)-(C₁- to C₄)-alkyl radical, and

G¹⁴ is a hydrogen or halogen atom, a C₁- to C₄-alkyl radical, a C₁- to C₄-monohydroxyalkyl radical, a C₂- to C₄-polyhydroxyalkyl radical, a (C₁- to C₄) -alkoxy-(C₁- to C₄)-alkyl radical, a C₁-to C₄-aminoalkyl radical or a C₁- to C₄-cyanoalkyl radical,

G15 is hydrogen, a C₁- to C₄-alkyl radical, a C₁to C₄-monohydroxyalkyl radical, a C₂- to C₄-polyhydroxyalkyl radical, a phenyl radical or a benzyl radical, and

G16 is hydrogen or a halogen atom.

The substituents used in formula (III) are defined according to the invention analogously to the above statements.

Preferred p-aminophenols of the formula (III) are, in particular, p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2-(2-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4amino-2-fluorophenol, 4-amino-2-chlorophenol, 2,6-dichloro-4-aminophenol, 4-amino-2-((diethylamino)methyl)phenol and their physiologically compatible salts.

Very particularly preferred compounds of the formula (III) are p-aminophenol, 4-amino-3-methylphenol, 4-amino-2-chlorophenol, 4-amino-2,6-dichlorophenol, 4-amino-2-aminomethylphenol and 4-amino-2-((diethylamino)methyl)phenol.

The developer component can also be chosen from o-aminophenol and its derivatives, such as, for example, 2-amino-4-methylphenol or 2-amino-4-chlorophenol.

In addition, the developer component can be chosen from heterocyclic developer components, such as, for example, the pyridine, pyrimidine, pyrazole, pyrazolepyrimidine derivatives and their physiologically compatible salts.

Preferred pyridine derivatives are, in particular, the compounds described in patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine.

Preferred pyrimidine derivatives are, in particular, the compounds described in German patent DE 23 59 399, Japanese laid-open specification JP-A2-02/019576 or in the laid-open specification WO 96/15765, such as 2,4,5,6-tetraaminopyrimidine, 4-hydroxy-2,5,6-triaminopyrimidine, 2-hydroxy-4,5,6-triaminopyrimidine, 2-di-methylamino-4,5,6-triaminopyrimidine, 2,4-dihydroxy-5,6-diaminopyrimidine and 2,5,6-triaminopyrimidine.

Preferred pyrazole derivatives are, in particular, the compounds described in patent DE 38 43 892, DE 41 33 957 and laid-open specifications WO 94/08969, WO 94/08970, EP 0 740 931 and DE 195 43 988, such as 4,5-diamino-1-methylpyrazole, 4,5-diamino-1-(β-hydroxy-ethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-di-amino-1-methyl-4-methylaminopyrazole and 3,5-diamino-4(β-hydroxyethyl)amino-1-methylpyrazole.

Preferred pyrazolepyrimidine derivatives are, in particular, the derivatives of pyrazole-[1,5-a]pyrimidine of the following formula (IV) and tautomeric forms thereof, where a tautomeric equilibrium exists:

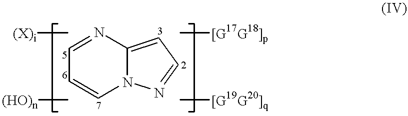

where:

G¹⁷, G¹⁸, G¹⁹, and G²⁰, independently of one another, are a hydrogen atom, a C₁- to C₄-alkyl radical, an aryl radical, a C₁- to C₄-hydroxyalkyl radical, a C₂- to C₄-polyhydroxyalkyl radical, a (C₁- to C₄)-alkoxy-(C₁to C₄)-alkyl radical, a C₁to C₄-aminoalkyl radical which may optionally be protected by an acetylureide or sulfonyl radical, a (C₁- to C₄)-alkylamino-(C₁- to C₄)-alkyl radical, a di[(C₁- to C₄) alkyl]-(C₁- to C₄)-aminoalkyl radical, where the dialkyl radicals optionally form a carbon cycle or a heterocycle having 5 or 6 chain members, a C₁- to C₄-hydroxyalkyl or a di(C₁-to C₄) - [hydroxyalkyl]-(C₁to C₄) -aminoalkyl radical,

the X radicals, independently of one another, are a hydrogen atom, a C₁- to C₄-alkyl radical, an aryl radical, a C₁to C₄-hydroxyalkyl radical, a C₂- to C₄-polyhydroxyalkyl radical, a C₁- to C₄-aminoalkyl radical, a (C₁- to C₄)-alkylamino-(C₁to C₄)-alkyl radical, a di[(C₁- to C₄)alkyl]-(C₁- to C₄)amino-alkyl radical, where the dialkyl radicals optionally form a carbon cycle or a heterocycle having 5 or 6 chain members, a C₁- to C₄-hydroxyalkyl or a di(C₁to C₄-hydroxyalkyl)-aminoalkyl radical, an amino radical, a C₁to C₄-alkyl or di(C₁to C₄-hydroxyalkyl)-amino radical, a halogen atom, a carboxylic acid group or a sulfonic acid group,

i has the value 0, 1, 2 or 3,

p has the value 0 or 1,

q has the value 0 or 1 and

n has the value 0 or 1,

with the proviso that

the sum p+q is not equal to 0,

if p+q is 2, n has the value 0 and the groups NG¹⁷G¹⁸ and NG¹⁹G²⁰ occupy positions (2,3); (5,6); (6,7); (3,5) or (3,7);

if p+q is 1, n has the value 1 and the groups NG¹⁷G⁸ (or NG¹⁹G²⁰) and the group OH occupy positions (2,3); (5,6); (6,7); (3,5) or (3,7).

The substituents used in formula (IV) are defined according to the invention analogously to the above statements.

If the pyrazole-[1,5-a]pyrimidine of the above formula (IV) contains a hydroxyl group on one of the positions 2, 5 or 7 of the ring system, a tautomeric equilibrium exists which is shown, for example, in the scheme below:

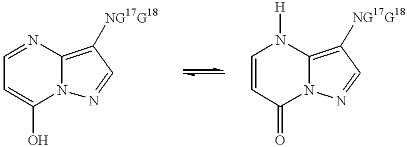

Of the pyrazole-[1,5-a]pyrimidines of the above formula (IV), mention may be made in particular of:

pyrazole-[1,5-a]pyrimidine-3,7-diamine;

2,5-dimethylpyrazole-[1,5-a]pyrimidine-3,7-diamine;

pyrazole-[1,5-a]pyrimidine-3,5-diamine;

2,7-dimethylpyrazole-[1,5-a]pyrimidine-3,5-diamine;

3-aminopyrazole-[1,5-a]pyrimidin-7-ol;

3-aminopyrazole-[1,5-a]pyrimidin-5-ol;

2-(3-aminopyrazole-[1,5-a]pyrimidin-7-ylamino)ethanol;

2-(7-aminopyrazole-[1,5-a]pyrimidin-3-ylamino)ethanol;

2-[(3-aminopyrazole-[1,5-a]pyrimidin-7-yl)-(2-hydroxyethyl)amino]ethanol;

2-[(7-aminopyrazole-[1,5-a]pyrimidin-3-yl)-(2-hydroxyethyl)amino]ethanol;

5,6-dimethylpyrazole-[1,5-a]pyrimidine-3,7-diamine;

2,6-dimethylpyrazole-[1,5-a]pyrimidine-3,7-diamine;

2,5,N7,N7-tetramethylpyrazole-[1,5-a]pyrimidine-3,7-diamine;

and their physiologically compatible salts and their tautomeric forms if a tautomeric equilibrium is present.

The pyrazole-[1,5-a]pyrimidines of the above formula (IV) can, as described in the literature, be prepared by cyclization starting from an aminopyrazole or from hydrazine.

The coupler components used are usually m-phenylene-diamine derivatives, naphthols, resorcinol and resorcinol derivatives, pyrazolones and m-aminophenol derivatives. Suitable coupler substances are, in particular, 1-naphthol, 1,5-, 2,7- and 1,7-dihydroxy-naphthalene, 5-amino-2-methylphenol, m-aminophenol, resorcinol, resorcinol monomethyl ether, m-phenylene-diamine, 1-phenyl-3-methylpyrazolone, 2,4-dichloro-3-aminophenol, 1,3-bis(2,4-diam-5-inophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol and 2-methyl-4-chloro-5-aminophenol.

Preferred coupler components according to the invention are:

m-aminophenol and its derivatives, such as, for example, 5-amino-2-methylphenol, 3-amino-2-chloro-6-methylphenol, 2-hydroxy-4-aminophenoxyethanol, 2,6-dimethyl-3-aminophenol, 3-trifluoroacetyl-amino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, N-cyclopentyl-3-aminophenol, 1, 3-dihydroxy-5- (methylamino) benzene, 3-(ethylamino)-4-methylphenol and 2,4-dichloro-3-aminophenol,

o-aminophenol and its derivatives,

m-diaminobenzene and its derivatives, such as, for example, 2,4-diaminophenoxyethanol, 1,3-bis (2,4-diaminophenoxy)propane, 1-methoxy-2-amino-4(2′-hydroxyethylamino)benzene, 1,3-bis(2,4-diaminophenyl)propane, 2,6-bis(2-hydroxyethylamino)-1-methylbenzene and 1-amino-3-bis(2′-hydroxyethyl)aminobenzene,

o-diaminobenzene and its derivatives, such as, for example, 3,4-diaminobenzoic acid and 2,3-diamino-1-methylbenzene,

di- and trihydroxybenzene derivatives, such as, for example, resorcinol, resorcinol monomethyl ether, 2-methylresorcinol, 5-methylresorcinol, 2,5-dimethylresorcinol, 2-chlororesorcinol, 4-chlororesorcinol, pyrogallol and 1,2,4-trihydroxybenzene,

pyridine derivatives, such as, for example, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine,

naphthalene derivatives, such as, for example, 1- naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene,

morpholine derivatives, such as, for example, 6-hydroxybenzomorpholine and 6-aminobenzomorpholine,

quinoxaline derivatives, such as, for example, 6-methyl-1,2,3,4-tetrahydroquinoxaline,

pyrazole derivatives, such as, for example, 1-phenyl-3-methylpyrazol-5-one,

indole derivatives, such as, for example, 4-hydroxyindole, 6-hydroxyindole and 7-hydroxyindole,

pyrimidine derivatives, such as, for example, 4,6-diaminopyrimidine, 4-amino-2,6-dihydroxypyrimidine, 2,4-diamino-6-hydroxypyrimidine, 2,4,6-tri-hydroxypyrimidine, 2-amino-4-methylpyrimidine, 2-amino-4-hydroxy-6-methylpyrimidine and 4,6-di-hydroxy-2-methylpyrimidine, or

methylenedioxybenzene derivatives, such as, for example, 1-hydroxy-3,4-methylenedioxybenzene, 1-amino-3,4-methylenedioxybenzene and 1-(2′-hydroxyethyl)amino-3,4-methylenedioxybenzene.

Particularly preferred coupler components are 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol and 2,6-dihydroxy-3,4-dimethylpyridine.

It is not necessary that the oxidation dye precursors or the substantive dyes are each uniform compounds. Rather, as a consequence of the preparation processes for the individual dyes, the hair dyeing agents according to the invention may also comprise further components in lesser amounts, provided these do not adversely affect the coloring result or do not have to be excluded for other reasons, e.g. toxicological reasons.

With regard to the dyes which can be used in the hair dyeing and tinting agents according to the invention, reference is also expressly made to the monograph Ch. Zviak, The Science of Hair Care, chapter 7 (pages 248-250; substantive dyes) and chapter 8, pages 264-267; oxidation dye precursors), published as volume 7 of the series “Dermatology” (Ed.: Ch., Culnan and H. Maibach), Verlag Marcel Dekker Inc., New York, Basle, 1986, and the “European Inventory of Cosmetic Raw Materials”, published by the European Community, obtainable in floppy disk form from the Bundesverband Deutscher Industrie- und Handelsunternehmen fur Arzneimittel, Reformwaren und Körperflegemittel e.V. [Federal Association of German Industrial and Commercial Enterprises for Medicaments, Health Goods and Body Care Products], Mannheim.

The oxidation dye precursors are preferably present in the agents according to the invention in amounts of from 0.01 to 20% by weight, preferably 0.5 to 5% by weight, in each case based on the overall agent.

As precursors of nature-analogous dyes, preference is given to using those indoles and indolines which have at least one hydroxyl or amino group, preferably as substituent on the 6-membered ring. These groups can carry further substituents, e.g. in the form of an etherification or esterification of the hydroxyl group or an alkylation of the amino group.

Particularly suitable precursors of nature-analogous hair dyes are derivatives of 5,6-dihydroxyindoline of the formula (Va),

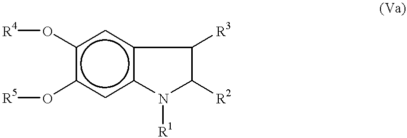

in which, independently of one another,

R¹ is hydrogen, a C₁-C₄-alkyl group or a C₁-C₄-hydroxyalkyl group,

R² is hydrogen or a —COOH group, where the —COOH group may also be present as a salt with a physiologically compatible cation,

R³ is hydrogen or a C₁-C₄-alkyl group,

R⁴ is hydrogen, a C₁-C₄-alkyl group or a group —CO—R⁶ in which R⁶ is a C₁-C₄-alkyl group, and

R⁵ is one of the groups given under R⁴, and physiologically compatible salts of these compounds with an organic or inorganic acid.

Particularly preferred derivatives of indoline are 5,6-dihydroxyindoline, N-methyl-5,6-dihydroxyindoline, N-ethyl-5,6-dihydroxyindoline, N-propyl-5,6-dihydroxyindoline, N-butyl-5, 6-dihydroxyindoline, 5, 6-dihydroxyindoline-2-carboxylic acid, and 6-hydroxyindoline, 6-aminoindoline and 4-aminoindoline.

Within this group, particular attention is drawn to N-methyl-5,6-dihydroxyindoline, N-ethyl-5,6-dihydroxyindoline, N-propyl-5,6-dihydroxyindoline, N-butyl-5,6-dihydroxyindoline and, in particular, 5,6-dihydroxyindoline.

Highly suitable as precursors of nature-analogous hair dyes are also derivatives of 5,6-dihydroxyindole of the formula (Vb),

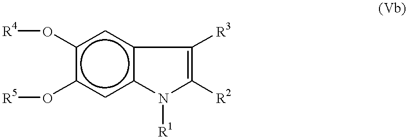

in which, independently of one another,

R¹ is hydrogen, a C₁-C₄-alkyl group or a C₁-C₄-hydroxyalkyl group,

R² is hydrogen or a —COOH group, where the —COOH group can also be in the form of a salt with a physiologically compatible cation,

R³ is hydrogen or a C₁-C₄-alkyl group,

R⁴ is hydrogen, a C₁-C₄-alkyl group or a group —CO—R⁶, in which R⁶ is a C₁-C₄-alkyl group, and

R⁵ is one of the groups given under R⁴,

and physiologically compatible salts of these compounds with an organic or inorganic acid.

Particularly preferred derivatives of indole are 5,6-dihydroxyindole, N-methyl-5, 6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole.

Within this group, particular attention is drawn to N-methyl-5,6-dihydroxyindole, N-ethyl-5,6-dihydroxyindole, n-propyl-5,6-dihydroxyindole, N-butyl-5,6-dihydroxyindole and, in particular, 5,6-dihydroxyindole.

In the dyeing agents used for the purposes of the method according to the invention, the indoline and indole derivatives may be used either as free bases or in the form of their physiologically compatible salts with inorganic or organic acids, e.g. the hydrochlorides, the sulfates and hydrobromides. The indole or indoline derivatives are present therein usually in amounts of from 0.05-10% by weight, preferably 0.2-5% by weight.

In a further embodiment, it may be preferred according to the invention to use the indoline or indole derivative in hair dyeing agents in combination with at least one amino acid or one oligopeptide. The amino acid is advantageously an α-amino acid; very particlarly preferred α-amino acids are arginine, ornithine, lysine and histidine, in particular arginine.

In addition to the dye precursors, the dyeing agents according to the invention can comprise substantive dyes for further tinting. These are customarily chosen from nitrophenylenediamines, nitroaminophenols, azo dyes, anthraquinones or indophenols. Preferred substantive dyes are the compounds known under the international names or tradenames HC Yellow 2, HC Yellow 4, HC Yellow 5, HC Yellow 6, Basic Yellow 57, HC Orange 1, Disperse Orange 3, HC Red 1, HC Red 3, HC Red 13, HC Red BN, Basic Red 76, HC Blue 2, HC Blue 12, Disperse Blue 3, Basic Blue 7, Basic Blue 26, Basic Blue 99, HC Violet 1, Disperse Violet 1, Disperse Violet 4, Basic Violet 2, Basic Violet 14, Acid Violet 43, Disperse Black 9, Acid Black 52, Basic Brown 16 and Basic Brown 17, and 1,4-bis(β-hydroxyethyl)amino-2-nitrobenzene, 3-nitro-4-(β-hydroxyethyl)aminophenol, 4-amino-2-nitrodiphenylamine-2′-carboxylic acid, 6-nitro-1,2,3,4-tetrahydroquinoxaline, 2-hydroxy-1,4-naphthoquinone, hydroxyethyl-2-nitrotoluidine, picramic acid, 2-amino-6-chloro-4-nitrophenol, 4-ethylamino-3-nitrobenzoic acid and 2-chloro-6-ethylamino-1-hydroxy-4-nitrobenzene. The agents of the invention according to this embodiment preferably comprise the substantive dyes in an amount of from 0.01 to 20% by weight, based on the overall dyeing agent.

The preparations according to the invention can also comprise naturally occurring dyes, such as, for example, henna red, henna neutral, henna black, camomile blossom, sandalwood, black tea, buckthorn bark, sage, logwood, madder root, catechu, sedre and alkanna root.

In addition to the oxidase system according to the invention, the dyeing agents according to the invention can also comprise further enzymes, such as, for example, laccases, peroxidases or oxidases with their corresponding substrates. Preferred oxidases are, for example, choline oxidase, glucose oxidase, alcohol oxidase, pyruvate oxidase, oxalate oxidase, cholesterol oxidase, uricase, lactate oxidase, xanthine oxidase, pyranose oxidase, glycerol oxidase and galactose oxidase. For the purposes of this embodiment, particular preference is given to agents which, in addition to the phenol-oxidizing enzyme, also comprise uricase, glucose oxidase and/or xanthine oxidase and their respective substrates.

For the preparation of the dyeing agents according to the invention, the dye precursors can be incorporated into a suitable hydrous carrier. For the purpose of hair dyeing, such carriers are, for example, creams, emulsions, gels and also sulfactant-containing foaming solutions, e.g. shampoos, foam aerosols or other preparations which are suitable for use on the hair.

The dyeing agents according to the invention can also comprise all active ingredients, additives and auxiliaries known for such preparations. In many cases, the dyeing agents comprise at least one surfactant, both anionic and also zwitterionic, ampholytic, nonionic and cationic surfactants being suitable in principle. The person skilled in the art can test any influence of the various surfactants on the activity of the enzyme system according to the invention where necessary by simple preliminary experiments.

In a preferred embodiment of the present invention, a combination of anionic and nonionic surfactants or a combination of anionic and amphoteric surfactants is used in the compositions for the dyeing of keratin fibers. However, in individual cases, it has proven advantageous to choose the surfactants from amphoteric or nonionic surfactants since these generally influence the dyeing process according to the invention to a lesser extent.

Suitable anionic surfactants in preparations according to the invention are all anionic surface-active substances suitable for use on the human body. These are characterized by a solubilizing, anionic group, such as, for example, a carboxylate, sulfate, sulfonate or phosphate group, and a lipophilic alkyl group having about 10 to 22 carbon atoms. Additionally, glycol or polyglycol ether groups, ester, ether and amide groups and hydroxyl groups may be present in the molecule. Examples of suitable anionic surfactants are, in each case in the form of the sodium, potassium and ammonium and the mono-, di- and trialkanolammonium salts having 2 or 3 carbon atoms in the alkanol group,

linear fatty acids having 10 to 22 carbon atoms (soaps),

ether carboxylic acids of the formula R—O—(CH₂—CH₂O)ₓ—CH₂—COOH, in which R is a linear -alkyl group having 10 to 22 carbon atoms and x 0 or 1 to 16,

acyl sarcosides having 10 to 18 carbon atoms in the acyl group,

acyl taurides having 10 to 18 carbon atoms in the acyl group,

acyl isethionates having 10 to 18 carbon atoms in the acyl group,

sulfosuccinic mono- and dialkyl esters having 8 to 18 carbon atoms in the alkyl group and sulfosuccinic monoalkyl polyoxyethyl esters having 8 to 18 carbon atoms in the alkyl group and 1 to 6 oxyethyl groups,

linear alkanesulfonates having 12 to 18 carbon atoms,

linear alpha-olefinsulfonates having 12 to 18 carbon atoms,

alpha-sulfo fatty acid methyl esters of fatty acids having 12 to 18 carbon atoms,

alkyl sulfates and alkyl polyglycol ether sulfates of the formula R—O(CH₂—CH₂O)ₓ—SO₃H, in which R is preferably a linear alkyl group having 10 to 18 carbon atoms and x=0 or 1 to 12,

mixtures of surface-active hydroxysulfonates according to DE-A-37 25 030,

sulfated hydroxyalkylpolyethylene and/or hydroxyalkylene propylene glycol ethers according to DE-A-37 23 354,

sulfonates of unsaturated fatty acids having 12 to 24 carbon atoms and 1 to 6 double bonds according to DE-A-39 26 344,

esters of tartaric acid and citric acid with alcohols, which represent addition products of about 2-15 molecules of ethylene oxide and/or propylene oxide with fatty alcohols having 8 to 22 carbon atoms.

Preferred anionic surfactants are alkyl sulfates, alkyl polyglycol ether sulfates and ether carboxylic acids having 10 to 18 carbon atoms in the alkyl group and up to 12 glycol ether groups in the molecule, and, in particular, salts of saturated and in particular unsaturated C₈-C₂₂-carboxylic acids, such as oleic acid, stearic acid, isostearic acid and palmitic acid.

Nonionogenic surfactants comprise, as hydrophilic group, e.g. a polyol group, a polyalkylene glycol ether group or a combination of polyol and polyglycol ether group. Such compounds are, for example,

addition products of from 2 to 30 mol of ethylene oxide and/or 0 to 5 mol of propylene oxide with linear fatty alcohols having 8 to 22 carbon atoms, with fatty acids having 12 to 22 carbon atoms and with alkylphenols having 8 to 15 carbon atoms in the alkyl group,

C₁₂-C₂₂ fatty acid mono- and diesters of addition products of from 1 to 30 mol of ethylene oxide with glycerol,

C₈-C₂₂-alkyl mono- and oligoglycosides and ethoxylated analogs thereof, and

addition products of from 5 to 60 mol of ethylene oxide with castor oil and hydrogenated castor oil.

Preferred nonionic surfactants are alkyl polyglycosides of the general formula R¹O—(Z)ₓ. These compounds are available, for example, under the tradename Plantacare® from Henkel and are characterized by the following parameters.

The alkyl radical R¹ contains 6 to 22 carbon atoms and can either be linear or branched. Preference is given to primary linear radicals and aliphatic radicals methyl-branched in the 2-position. Such alkyl radicals are, for example, 1-octyl, 1-decyl, 1-lauryl, 1-myristyl, 1-cetyl and 1-stearyl. Particular preference is given to 1-octyl, 1-decyl, 1-lauryl, 1-myristyl. If “oxo alcohols” are used as starting materials, compounds with an odd number of carbon atoms in the alkyl chain predominate.

The alkyl polyglycosides which can be used according to the invention can comprise, for example, only one specific alkyl radical R¹. However, these compounds are usually prepared starting from natural fats and oils or mineral oils. In this case, mixtures corresponding to the starting compounds or corresponding to the respective work-up of these compounds are present as alkyl radicals R.

Particular preference is given to alkyl polyglycosides in which R¹ consists

essentially of C₈- and C₁₀-alkyl groups,

essentially of C₁₂- and C₁₄-alkyl groups,

essentially of C₈- to C₁₆-alkyl groups, or

essentially of C₁₂- to C₁₆-alkyl groups.

It is possible to use any mono- or oligosaccharides as sugar building block Z. Sugars containing 5 or 6 carbon atoms and the corresponding oligosaccharides are usually used. Such sugars are, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred.

The alkyl polyglycosides which can be used according to the invention contain, on average, 1.1 to 5 sugar units. Alkyl polyglycosides with x values of from 1.1 to 1.6 are preferred. Very particular preference is given to alkyl glycosides in which x is 1.1 to 1.4.

Apart from their surfactant action, the alkyl glycosides can also serve to improve the fixing of fragrance components on the hair. Thus, in cases where the effect of the perfume oil on the hair beyond the duration of the hair treatment is desired, the person skilled in the art will preferably have recourse to this class of substance as a further ingredient of the preparations according to the invention. An alkyl glucoside which is particularly preferred according to the invention is the commercial product Plantacare® 1200G.

The alkoxylated homologs of said alkyl polyglycosides can also be used according to the invention. These homologs can contain, on average, up to 10 ethylene oxide and/or propylene oxide units per alkyl glycoside unit. In addition, zwitterionic surfactants can be used, in particular as cosurfactants. “Zwitterionic surfactants” is the term used to describe those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinate, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl hydroxyethyl-carboxymethylglycinate. A preferred zwitterionic surfactant is the fatty acid amide derivative known under the INCI name Cocamidopropyl Betaine.

Ampholytic surfactants are likewise suitable, in particular as cosurfactants. Ampholytic surfactants are understood as meaning those surface-active compounds which, apart from a C₈-C₁₈-alkyl or acyl group in the molecule, contain at least one free amino group and at least one -COOH or -SO₃H group and are able to form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂-₁₈-acylsarcosine.

According to the invention, the cationic surfactants used are, in particular, those of the quaternary ammonium compound type, the ester quat type and the amidoamine type.

Preferred quaternary ammonium compounds are ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, and the imidazolium compounds known under the INCI names Quaternium-27 and Quaternium-83. The long alkyl chains of the abovementioned surfactants preferably have 10 to 18 carbon atoms.

The ester quats are known substances which contain both at least one ester function and also at least one quaternary ammonium group as structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold, for example, under the tradenames Stepantex®, Dehyquart® and Armocare®. The products Armocare® VGH-70, an N,N-bis(2-palmitoyloxyethyl)dimethylammonium chloride, and Dehyquart® F-75 and Dehyquart® AU-35 are examples of such ester quats.

The alkylamidoamines are usually prepared by amidation of natural or synthetic fatty acids and fatty acid fractions with dialkylaminoamines. A particularly suitable compound from this group of substances according to the invention is the stearamidopropyldimethylamine available commercially under the name Tegoamid® S 18.

Further cationic surfactants which can be used according to the invention are the quaternized protein hydrolyzates.

Likewise suitable according to the invention are cationic silicone oils, such as, for example, the commercially available products Q2-7224 (manufacturer: Dow Corning; a stabilized trimethylsilylamodimethicone), Dow Corning 929 emulsion (comprising a hydroxyl-amino-modified silicone, which is also referred to as amodimethicones), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80).

An example of a quaternary sugar derivative which can be used as cationic surfactant is the commercial product Glucquat® 100, according to INCI nomenclature a “Lauryl Methyl Gluceth-10 Hydroxypropyl Dimonium Chloride”.

The compounds containing alkyl groups used as surfactant may in each case be uniform substances. However, it is generally preferred to start from native vegetable or animal raw materials in the preparation of these substances, meaning that substance mixtures with varying alkyl chain lengths, depending on the raw material in question, are obtained.

In the case of the surfactants which are addition products of ethylene oxide and/or propylene oxide with fatty alcohols or derivatives of these addition products, it is possible to use either products with a “normal” homolog distribution or those with a narrowed homolog distribution. “Normal” homolog distribution is understood as meaning here mixtures of homologs obtained during the reaction of fatty alcohol and alkylene oxide using alkali metals, alkali metal hydroxides or alkali metal alkoxides as catalysts. By contrast, narrowed homolog distributions are obtained when, for example, hydrotalcites, alkaline earth metal salts of ether carboxylic acids, alkaline earth metal oxides, hydroxides or alkoxides are used as catalysts. The use of products with narrowed homolog distribution may be preferred.

Furthermore, the agents according to the invention preferably comprise at least one alkalizing agent. Preferred alkalizing agents are ammonia, monoethanolamine, sodium hydroxide, potassium hydroxide and, in particular, arginine, lysine and histidine.

Furthermore, the dyeing agents according to the invention may preferably also comprise a conditioning active ingredient chosen from the group formed by cationic surfactants, cationic polymers, alkylamidoamines, paraffin oils, vegetable oils and synthetic oils. With regard to the cationic surfactants, reference may be made to the above statements.

Cationic polymers may be preferred as conditioning active ingredients. These are usually polymers which contain a quaternary nitrogen atom, for example in the form of an ammonium group.

Preferred cationic polymers are, for example,

quaternized cellulose derivatives, as are commercially available under the names Celquat® and Polymer JR®. The compounds Celquat® H 100, Celquat® L 200 and Polymer JR®400 are preferred quaternized cellulose derivatives.

polymeric dimethyldiallylammonium salts and copolymers thereof with acrylic acid and esters and amides of acrylic acid and methacrylic acid.

The products commercially available under the names Merquat® 100 (poly(dimethyldiallylammonium chloride)), Merquat® 550 (dimethyldiallylammonium chloride-acrylamide copolymer) and Merquat® 280 (dimethyldiallylammonium chloride/acrylic acid copolymer are examples of such cationic polymers.

copolymers of vinylpyrrolidone with quaternized derivatives of dialkylaminoacrylate and -methacrylate, such as, for example, vinylpyrrolidone-dimethylaminomethacrylate copolymers quaternized with diethyl sorbate. Such compounds are commercially available under the names Gafquat® 734 and Gafquat®755.

vinylpyrrolidone-methoimidazolinium chloride copolymers, as supplied under the name Luviquat®.

quaternized polyvinyl alcohol

and the polymers known under the names

polyquaternium 2,

polyquaternium 17,

polyquaternium 18 and

polyquaternium 27 with quaternary nitrogen atoms in the polymer main chain.

Particular preference is given to cationic polymers of the four first-named groups and polyquaternium-2, and very particular preference is given to polyquaterium-2, polyquaternium-10 and polyquaternium-22. Of the compounds known as polyquaternium-2, preference is given in particular to the commerical product Mirapol® A-15.

Also suitable as conditioning active ingredients are silicone oils, in particular dialkyl- and alkylarylsiloxanes, such as, for example, dimethylpolysiloxane and methylphenylpolysiloxane, and alkoxylated and quaternized analogs thereof. Examples of such silicones are the products sold by Dow Corning under the names DC 190, DC 200, DC 344, DC 345 and DC 1401, and the commercial products Q2-7224 (manufacturer: Dow Corning; a stabilized trimethylsilylamodimethicone), Dow Corning® 929 emulsion (comprising a hydroxylamino-modified silicone which is also referred to as Amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxane, quaternium-80).

As conditioning active ingredients it is also possible to use paraffin oils, synthetically prepared oligomeric alkenes and vegetable oils such as jojoba oil, sunflower oil, orange oil, almond oil, wheatgerm oil and peach kernel oil.

Likewise suitable hair-conditioning compounds are phospholipids, for example soybean lecithin, egg lecithin and cephalins.

Furthermore, the preparations used according to the invention preferably comprise at least one oil component.

Oil components suitable according to the invention are, in principle, all water-insoluble oils and fatty substances and mixtures thereof with solid paraffins and waxes. Water-insoluble is used according to the invention to define substances whose solubility in water is less than 0.1% by weight at 20° C. The melting point of the individual oil or fat components is preferably below about 40° C. Oil and fat components which are liquid at room temperature, i.e. below 25° C., may be particularly preferred according to the invention. However, if two or more oil and fat components and optionally solid paraffins and waxes are used, it also usually suffices for the mixture of the oil and fat components and optionally paraffins and waxes to satisfy these conditions.

A preferred group of oil components are vegetable oils. Examples of such oils are sunflower oil, olive oil, soybean oil, rapeseed oil, almond oil, jojoba oil, orange oil, wheatgerm oil, peach kernel oil and the liquid fractions of coconut oil.

Also suitable, however, are other triglyceride oils, such as the liquid fractions of beef tallow and synthetic triglyceride oils.

A further, particularly preferred group of compounds which can be used according to the invention as oil component are liquid paraffin oils and synthetic hydrocarbon and di-n-alkyl ethers with a total of between 12 and 36 carbon atoms, in particular 12 and 24 carbon atoms, such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert-butyl ether, diisopentyl ether, di-3-ethyldecyl ether, tert-butyl n-octyl ether, isopentyl n-octyl ether and 2-methylpentyl n-octyl ether. The compounds 1,3-di(2-ethylhexyl)cyclohexane (Cetiol® S) and di-n-octyl ether (Cetiol® OE), which are available as commercial products, may be preferred.

Oil components which can likewise be used according to the invention are fatty acid and fatty alcohol esters. Preference is given to the monoesters of fatty acids with alcohols having 3 to 24 carbon atoms. This group of substances includes the products of the esterification of fatty acids having 6 to 24 carbon atoms, such as, for example, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid, and technical-grade mixtures thereof which are produced, for example, during the pressurized cleavage of natural fats and oils, the oxidation of aldehydes from the Roelen oxo synthesis or the dimerization of unsaturated fatty acids, with alcohols such as, for example, isopropyl alcohol, caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, and technical-grade mixtures thereof which are produced, for example, during the high-pressure hydrogenation of technical-grade methyl esters based on fats and oils or aldehydes from the Roelen oxo synthesis, and as monomer fraction during the dimerization of unsaturated fatty alcohols. According to the invention, isopropyl myristate, C16-18-alkyl isononoate (Cetiol SN), 2-ethylhexyl stearate (Cetiol® 868), cetyl oleate, glycerol tricaprylate, coconut fatty alcohol caprate/caprylate and n-butyl stearate are particularly preferred.

Furthermore, dicarboxylic esters, such as di-n-butyl adipate, di(2-ethylhexyl) adipate, di(2-ethylhexyl) succinate and diisotridecyl acelate, and diol esters, such as ethylene glycol dioleate, ethylene glycol diisotridecanoate, propylene glycol di(2-ethyl hexanoate), propylene glycol diisostearate, propylene glycol dipelargonate, butanediol diisostearate, neopentylglycol dicaprylate also represent oil components which can be used according to the invention, as do complex esters, such as, for example, diacetyl glycerol monostearate.

Finally, it is also possible to use fatty alcohols having 8 to 22 carbon atoms as oil components effective according to the invention. The fatty alcohols may be saturated or unsaturated and linear or branched. For the purposes of the invention it is possible to use, for example, decanol, octanol, octenol, dodecenol, decenol, octadienol, dodecadienol, decadienol, oleyl alcohol, erucic alcohol, ricinoleic alcohol, stearyl alcohol, isostearyl alcohol, cetyl alcohol, lauryl alcohol, myristyl alcohol, arachidyl alcohol, caprylic alcohol, capric alcohol, linoleyl alcohol, linolenyl alcohol and behenyl alcohol, and Guerbet alcohols thereof, the intention being for this list to be exemplary and nonlimiting. However, the fatty alcohols originate from preferably natural fatty acids, it usually being possible to start from an isolation from the esters of the fatty acids by reduction. Those fatty alcohol fractions which are produced by reduction of naturally occurring triglycerides, such as beef tallow, palm oil, peanut oil, rapeseed oil, cottonseed oil, soybean oil, sunflower oil and linseed oil or fatty acid esters formed from their transesterification products with corresponding alcohols, and also a mixture of different fatty alcohols can likewise be used according to the invention.

The oil components are preferably used in amounts of from 0.05 to 10% by weight, in particular from 0.1 to 2% by weight, in the dyeing agents according to the invention.

In a preferred embodiment of the present invention, upon dissolution of the dyeing agent in water, a gel forms. For this purpose, thickeners such as agar agar, guar gum, alginates, xanthan gum, gum arabic, karaya gum, carob seed grain, linseed gums, dextrans, cellulose derivatives, e.g. methylcellulose, hydroxyalkylcellulose and carboxymethylcellulose, starch fractions and derivatives such as amylose, amylopectin and dextrins, clays, such as, for example, bentonite or fully synthetic hydrocolloids, such as e.g. polyvinyl alcohol, are added to the dyeing agent. Particularly preferred thickeners are xanthans, alginates and highly substituted carboxymethyl-celluloses.

Further active ingredients, auxiliaries and additives are, for example,

zwitterionic and amphoteric polymers, such as, for example, acrylamidopropyltrimethylammonium chloride/acrylate copolymers and octylacrylamide/methyl methacrylate/tert-butylaminoethyl methacrylate/2-hydroxypropyl methacrylate copolymers,

anionic polymers, such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butylacrylamide terpolymers,

structurants, such as maleic acid and lactic acid,

protein hydrolyzates, in particular the hydrolyzates of elastin, collagen, keratin, milk protein, soybean protein and wheat protein, the condensation products thereof with fatty acids, and quaternized protein hydrolyzates,

perfume oils, dimethyl isosorbide and cyclodextrins,

solvents and solubility promoters, such as ethanol, isopropanol, ethylene glycol, propylene glycol, glycerol and diethylene glycol,

fiber structure-improving active ingredients, in particular mono-, di- and oligosaccharides, such as, for example, glucose, galactose, fructose, fruit sugars and lactose,

quaternized amines, such as methyl-1-alkylamidoethyl-2-alkylimidazolinium methosulfate

antifoams, such as silicones,

dyes for coloring the agent,

antidandruff active ingredients, such as piroctone olamine, zinc omadine and climbazole,

light protection agents, in particular derivatized benzophenones, cinnamic acid derivatives and triazines,

substances for adjusting the pH, such as, for example, customary acids, in particular food acids and bases

active ingredients, such as allantoin, pyrrolidonecarboxylic acids and salts thereof, and bisabolol,

vitamins, provitamins and vitamin precursors, in particular those of groups A, B₃, Bs, B₆, C, E, F and H,

plant extracts, such as the extracts from green tea, oak bark, stinging nettle, hamamelis, hops, camomile, burdock, horsetail, hawthorn, lime blossom, almond, aloe vera, spruce needle, horse chestnut, sandalwood, juniper, coconut, mango, apricot, lime, wheat, kiwi, melon, orange, grapefruit, sage, rosemary, birch, mallow, lady's smock, wild thyme, yarrow, thyme, balm, restharrow, coltsfoot, marshmallow, meristem, ginseng and root ginger,

cholesterol,

bodying agents, such as sugar esters, polyolesters or polyol alkyl ethers,

fats and waxes, such as spermaceti, beeswax, montan wax and paraffins,

fatty acid alkanolamides,

complexing agents, such as EDTA, NTA, β-alaninediacetic acid and phosphonic acids,

swelling and penetration substances, such as glycerol, propylene glycol monoethyl ether, carbonates, hydrogencarbonates, guanidines, ureas and primary, secondary and tertiary phosphates,

opacifiers, such as latex, styrene/PVP and styrene/acrylamide copolymers,

pearlizing agents, such as ethylene glycol mono- and distearate, and PEG-3 distearate,

pigments,

stabilizing agents for hydrogen peroxide and other oxidizing agents,

propellants, such as propane/butane mixtures, N₂O, dimethyl ether, CO₂ and air,

antioxidants.

With regard to further optional components and the amounts of these components used, reference is expressly made to the relevant handbooks known to the person skilled in the art, e.g. Kh. Schrader, Gundlagen und Rezepturen der Kosmetika [Cosmetic Formulations and Bases], 2nd edition, Hüthig Buch Verlag, Heidelberg, 1989.

Expediently, the enzyme preparation is mixed with the preparation of the dye precursors directly prior to dyeing the hair. The application temperatures can be in a range between 15 and 40° C. After a contact time of about 30 minutes, the hair dyeing agent is removed from the hair to be dyed by rinsing out. Post-washing with a shampoo is dispensed with if a carrier with a high content of surfactant, e.g. a coloring shampoo, has been used.

In a further embodiment of the present invention, it may be preferred to formulate the enzyme preparation free from antioxidants and/or complexing agents since these are able to block the action of the enzymes.

The examples below serve to illustrate the subject-matter of the invention in more detail.

### EXAMPLE 1

a) Enzyme Solution

Phenoloxidase was prepared from Stachybotrys bisbyi as follows:

Cultivation of the strain was carried out in a composition which is given in Table 1. This composition has a pH of 5.4 and was autoclaved.

Table 1: Composition for the Cultivation

| Component | Amount [g/l] |
|---|---|
| Glucose | 15 |
| N-Z-Soy Peptone [Sigma] | 3 |
| KH₂PO₄ | 0.6 |
| ZnSO₄.7H₂O | 0.001 |
| K₂HPO₄ | 0.04 |
| FeSO₄.7H₂O | 0.005 |
| MnSO₄.H₂O | 0.05 |
| MgSO₄.7H₂O | 0.05 |
| CuSO₄.5H₂O | 0.25 |
| Water | ad 1 l |

For this, 30 ml cultures were left for 14 days at a shaking frequency of 150 rpm and 30° C. After 14 days the cultures were centrifuged at 12,000 revolutions/minute.

The enzyme activity of the culture supernatant was determined according to the following test.

1.5 ml of aqueous sodium acetate solution (120 mM/l, adjusted to pH 5 with dilute sodium hydroxide solution) were treated with 0.2 ml of ABTS (2,2′-azinobis(3-ethylbenzthiazoline-6-sulfonic acid) and 0.3 ml of the centrifuged-off culture supernatant. The conversion of ABTS by the enzyme solution was determined as a decrease in the absorption at 420 nm over a period of 2 minutes in a spectrophotometer. The activity of the enzyme solution was expressed in AOD/min (OD=optical density). A culture supernatant with an enzyme activity of 8 [ΔOD/min] was used for the coloration below.

b) Coloring Cream:

A base cream of the composition given in Table 2 was prepared.

TABLE 2
Base cream:

| Component | Amount [g] |
|---|---|
| Lorol ® , techn.¹ | 6.5 |
| Hydrenol ® D² | 24.2 |
| Dehyton ® K³ | 20.0 |
| Texapon ® NSO⁴ | 32.0 |
| Bidist. Water | ad 100 |

¹C12-18-fatty alcohol (INCI name: Coconut Alcohol) (COGNIS)
²C16-C18-fatty alcohol (INCI name: Cetearyl alcohol) (COGNIS)
³Fatty acid amide with betaine structure (about 30% active substance; INCI name: Cocamidopropyl Betaine) (Henkel)
⁴Sodium salt of lauryl ether sulfate (about 27% active substance; INCI name: Sodium Laureth Sulfate) (COGNIS)

12.5 g of this base cream were made up to 24 g with bidist. water. The pH was then adjusted to 7 using 1.5% strength aqueous ammonia solution, and the cream was finished by topping up to 25 g with bidist. water.

For the preparation of the coloring preparation, this cream was heated with continuous stirring until it was liquid. The dissolved developer and coupler substances (0.0025 mmol of 4-aminophenol in water and 0.0025 mmol of 5-amino-2-methylphenol in propylene glycol) were then added, and the cream was stirred until homogeneous. During this operation, the cream cooled to about 30° C. For the colorations, 1 g of this coloring preparation was in each case mixed with

1 g of the enzyme-containing culture supernatant (B1) or

1 g of a 2% strength aqueous hydrogen peroxide solution (Cl) (comparative experiment with conventional oxidizing agent) or

1 g of bidist. water (C2) (comparative experiment without oxidizing agent).

The 2 g of this end preparation were applied to a hair tress (human hair natural white (Kerling), 0.5 g). After a contact time of 30 minutes at room temperature, the hair was rinsed with lukewarm water and dried in the air.

The assessment of the colorations obtained are given in Table 3:

TABLE 3
Coloring results

| | | | | Color strength |
|---|---|---|---|---|
| Experi- | Oxidizing | | ΔE | change [%] |
| ment | agent | Color¹ | value² | compared with V2 |
| B1 | Enzyme | Persian | 19.35 | 244 |
| | solution | orange | | |
| C1 | H₂O₂ | Gray- | 11.24 | 192 |
| | | orange | | |
| C2 | — | Pale | 0 | 100 (reference) |
| | | orange | | |

¹A. Kornerup and J.H. Wanscher, taschenlexikon der Farben [Pocket Lexicon of Colors], Muster-Schmidt-Verlag, 3rd edition 1981.
²According to DIN 5033 (part 3) of July 1992

The coloration of the tresses was measured by colorimetry at 4 measurement points using a Datacolor Text Flash instrument from Data Color International, the measurement results were evaluated according to formula (1) using the Data Color Tools QC software and summarized in the table below. The reference used was the coloration of a tress according to C2.

$$\frac{\frac{K}{{S}_{Sample}}}{\frac{K}{{S}_{Reference}}} * 100 = color strength [ \% ] \qquad ( 1 )$$

where

K=absorption coefficient

S=scattering coefficient

K/S=reflection coefficient

In addition, the ΔE value of the CIELAB color system was determined according to formula (2)

ΔE=\sqrt{(ΔL)²+(ΔA)²+(ΔB)²} (2)

### EXAMPLE 2

Further colorations were carried out analogously to Example 1. In this connection, 8 g of each of the coloring preparations 1-4 listed in Table 4 were mixed with 2 g of the culture supernatant of Stachybotrys bisbyi with an enzyme activity of 8 [ΔOD/min] . 2 g of this end preparation were then applied to human hair; the further procedure was as in Example 1.

TABLE 4

| Component [g] | 1 | 2 | 3 | 4 |
|---|---|---|---|---|
| Lorol ® , techn.-grade | 3.2 | 3.2 | 3.2 | 3.2 |
| Eumulgin ® B2⁵ | 0.5 | 0.5 | 0.5 | 0.5 |
| Hydrenol ® D | 12.1 | 12.1 | 12.1 | 12.1 |
| Dehyton ® K | 10.0 | 10.0 | 10.0 | 10.0 |
| Plantacare ® 1200⁶ | 3.0 | 3.0 | 3.0 | 3.0 |
| N-Acetylcysteine | — | — | 0.5 | 1.0 |
| Sodium citrate | 0.5 | 1.0 | — | — |
| 2,4,5,6-Tetraamino- | 0.025 | 0.02057 | — | — |
| pyrimidine sulfate | | | | |
| p-Tolylenediamine | 0.025 | 0.8910 | 0.6144 | 0.96 |
| sulfate | | | | |
| 3-Methyl-4-amino- | 0.6 | 0.43 | 0.0518 | — |
| phenol | | | | |
| 4-Chlororesorcinol | 0.0316 | 0.242 | 0.1753 | — |
| 2-Amino-3-hydroxy- | 0.5362 | — | — | — |
| pyridine | | | | |
| 1-(2′-Hydroxyethyl)- | 0.1 | — | — | — |
| amino-4-methyl-2- | | | | |
| nitrobenzene | | | | |
| 2-Methylresorcinol | — | 0.242 | 0.0722 | — |
| 2,7-Dihydroxynaph- | — | 0.6 | — | — |
| thalene | | | | |
| m-Aminophenol | — | — | 0.0293 | 0.0893 |
| Resorcinol | — | — | 0.1109 | — |
| 1,3-Bis(2,4-diamino- | — | — | — | 0.361 |
| phenoxy)propane · 4HCl | | | | |
| 2-Methylamino-3- | — | — | — | 0.1853 |
| amino-5-methoxy- | | | | |
| pyridine | | | | |
| Isopropanolamine | | ad pH 7.0 | — | ad pH 7.0 |
| Monoethanolamine | ad pH 7.0 | — | ad pH 7.0 | — |
| Bidist. Water | ← ad 100 g→ | | | |
| Coloration | Copper | Hazelnut | Pale | Black |
| | gold | | brown | |

⁵Cetylstearyl alcohol with about 20 EO units (INCI name: Ceteareth-20) (COGNIS)
⁶C₁₂₋₁₆-fatty alcohol 1,4-glucoside (about 50% active substance; INCI name: Lauryl Glucoside) (COGNIS)

### EXAMPLE 3

The following dyeing agents were prepared (unless indicated otherwise, all amounts are parts by weight).

| | |
|---|---|
| 1. Dyeing agent A | |
| Fatty alcohol mixture, C₁₂-C₁₈ | 5.25 |
| Eumulgin ® B2 | 0.4 |
| Plantacare ® 1200 | 3.0 |
| Dehyton ® K | 2.5 |
| Ammonium sulfate | 0.5 |
| Natrosol ® 250 HR | 1.0 |
| 4-Amino-2-((diethylamino)methyl)- | 2.67 |
| phenol dihydrochloride | |
| 2-Amino-3-hydroxypyridine | 1.1 |
| Ammonia (25% strength in water) | ad pH 8.5 |
| Water | ad 100 |
| 2. Dyeing agent B | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-aminomethylphenol | 0.002 |
| dihydrochloride | |
| 2-Amino-3-hydroxypyridine | 0.004 |
| 3-Methyl-4-aminophenol | 0.007 |
| 1,3-Bis(2,4-diaminophenoxy)propane | 0.0005 |
| tetrahydrochloride | |
| p-Tolylenediamine sulfate | 0.06 |
| Resorcinol | 0.01 |
| 3-Amino-2,4-dichlorophenol | 0.008 |
| hydrochloride | |
| 3-Aminophenol | 0.002 |
| 4-Chlororesorcinol | 0.01 |
| Ammonia (25% strength in water) | ad pH 8.5 |
| Water | ad 100 |
| 3. Dyeing agent C | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-chlorophenol | 0.8 |
| 2-Amino-3-hydroxypyridine | 0.1 |
| 3-Methyl-4-aminophenol | 0.3 |
| 5-amino-2-methylphenol | 0.07 |
| Resorcinol | 0.2 |
| 3-Aminophenol | 0.2 |
| 1-(2′-hydroxyethyl)-2,5-diamino- | 0.7 |
| benzene sulfate | |
| 4-Methylresorcinol | 0.1 |
| 5,6-Dihydroxyindole | 0.05 |
| Monoethanolamine | ad pH 8.5 |
| Water | ad 100 |
| 4. Dyeing agent D | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Aminophenol | 0.49 |
| 4-Amino-3-methylphenol | 0.55 |
| 2-amino-3-hydroxypyridine | 1.0 |
| p-Tolylenediamine sulfate | 0.2 |
| 6-Hydroxyindole | 0.1 |
| 5-((2′-Hydroxyethyl)amino)-2- | 0.1 |
| methylphenol | |
| 1,2,3,4-Tetrahydro-6- | 0.05 |
| nitroquinoxaline | |
| HO Yellow 5⁸ | 0.03 |
| HO Red 1⁹ | 0.02 |
| Sodium hydroxide | ad pH 8.5 |
| Water | ad 100 |
| 5. Dyeing agent E | |
| Fatty alcohol mixture, C₁₂-C₁₈ | 5.25 |
| Eumulgin ® E2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-aminophenol | 2.0 |
| dihydrochloride | |
| 2-Methylamino-3-amino-6- | 2.25 |
| methoxypyridine | |
| Arginine | ad pH 8.5 |
| Water | ad 100 |
| 6. Dyeing agent F | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-aminomethylphenol | 0.017 |
| dihydrochloride | |
| 2-Methylamino-3-amino-6- | 0.002 |
| methoxypyridine | |
| 1-(2′-Hydroxyethyl)-2,5-diamino- | 0.03 |
| benzene sulfate | |
| 1-Naphthol | 0.02 |
| 2-Methylresorcinol | 0.0006 |
| Lysine | ad pH 8.5 |
| Water | ad 100 |
| 7. Dyeing agent G | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-((diethylamino)methyl)- | 0.1 |
| phenol dihydrochloride | |
| 2-Methylamino-3-amino-6- | 0.002 |
| methoxypyridine | |
| 3-Methyl-4-aminophenol | 0.07 |
| 2,4,5,6-Tetraaminopyrimidine | 1.0 |
| sulfate | |
| p-Tolylenediamine sulfate | 1.1 |
| 3-Aminophenol | 0.006 |
| Resorcinol | 0.11 |
| 2-Methylresorcinol | 0.54 |
| 2,7-Dihydroxynaphthalene | 0.032 |
| 2-Amino-3-hydroxypyridine | 0.4 |
| 4-Amino-2-nitrodiphenylamine-2′- | 0.1 |
| carboxylic acid | |
| Ammonia (25% strength in water) | ad pH 8.5 |
| Water | ad 100 |
| 8. Dyeing agent H | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-chlorophenol | 1.0 |
| dihydrochloride | |
| 2-Methylamino-3-amino-6- | 1.7 |
| methoxypyridine | |
| 2-Amino-4-nitro-6-chlorophenol | 0.05 |
| HC Red BN¹⁰ | 0.2 |
| 5,6-Dihydroxyindoline hydrobromide | 0.1 |
| Ammonia (25% strength in water) | ad pH 3.5 |
| Water | ad 100 |
| 9. Dyeing agent I | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-((diethylamino)methyl)- | 1.25 |
| phenol dihydrochloride | |
| 4-Amino-2-aminomethylphenol | 1.0 |
| dihydrochloride | |
| 5-Amino-2-methylphenol | 1.23 |
| Ammonia (25% strength in water) | ad pH 3.5 |
| Water | ad 100 |
| 10. Dyeing agent J | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-((diethylamino)methyl)- | 0.5 |
| phenol dihydrochloride | |
| Bis(2-hydroxy-5- | 0.4 |
| aminophenyl)methane | |
| 5-Amino-2-methylphenol | 0.55 |
| p-Tolylenediamine sulfate | 0.3 |
| p-Phenylenediamine dihydrochloride | 0.2 |
| Resorcinol | 0.07 |
| 2,7-Dihydroxynaphthalene | 0.09 |
| 2-Methylresorcinol | 0.07 |
| o-Aminophenol | 0.03 |
| 5,6-Dihydroxyindoline hydrobromide | 0.05 |
| Ammonia (25% strength in water) | ad pH 8.5 |
| Water | ad 100 |
| 11. Dyeing agent K | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Ammonium sulfate | 0.5 |
| Natrosol ® 250 HR | 1.0 |
| 4-Amino-2-aminomethylphenol | 0.02 |
| dihydrochloride | |
| 5-Amino-2-methylphenol | 0.03 |
| 3-Methyl-4-aminophenol | 0.01 |
| 2,4,5,6-Tetraaminopyrimidine | 0.8 |
| sulfate | |
| p-Tolylenediamine sulfate | 0.09 |
| 3-Aminophenol | 0.004 |
| Resorcinol | 0.04 |
| 2-Methylresorcinol | 0.22 |
| 2,7-Dihydroxynaphthalene | 0.3 |
| 4-Amino-2-nitrodiphenylamine-2′- | 0.15 |
| carboxylic acid | |
| 1,2,3,4-Tetrahydro-E-nitroquinox- | 0.25 |
| aline | |
| Ammonia (25% strength in water) | ad pH 8.5 |
| Water | ad 100 |
| 12. Dyeing agent L | |
| Fatty alcohol mixture, C₁₂-C₁₈ | 5.25 |
| Eumulgin ® B2 | 0.4 |
| Plantacare ® 1200 | 3.0 |
| Dehyton K | 2.5 |
| Sodium sulfite | 0.1 |
| Ammonium sulfate | 0.5 |
| Natrosol ® 250 HR | 1.0 |
| 4-Amino-2-aminomethylphenol | 1.06 |
| dihydrochloride | |
| 4-Aminophenol | 0.55 |
| 5-Amino-2-methylphenol | 0.74 |
| 4-Hydroxyindole | 0.1 |
| 1,3-Bis(2,4-diaminophenoxy)propane | 0.3 |
| tetrahydrochloride | |
| 2-Methylamino-3-amino-6- | 0.1 |
| methoxypyridine dihydrochloride | |
| Ammonia (25% strength in water) | ad pH 8.5 |
| Water | ad 100 |
| 13. Dyeing agent M | |
| Hydrenol ® D | 8.0 |
| C12-C18 fatty alcohol | 2.0 |
| Akypo Soft ® 45 NV¹¹ | 10.0 |
| Eumulgin ® B1¹² | 0.5 |
| Eumulgin ® B2 | 0.5 |
| 5-Amino-2-methylphenol | 0.02 |
| 3-Amino-2-chloro-6-methylphenol | 0.36 |
| Bis(2,4-diaminophenoxy)propane | 0.0002 |
| tetrahydrochloride | |
| 3-Methyl-4-aminophenol | 0.02 |
| 4-Amino-2-aminomethylphenol | 0.40 |
| dihydrochloride | |
| p-Tolylenediamine sulfate | 0.34 |
| N,N-Bis(2-hydroxyethyl)-p- | 0.48 |
| phenylenediamine sulfate | |
| Resorcinol | 0.33 |
| m-Aminophenol | 0.001 |
| 2-Amino-3-hydroxypyridine | 0.009 |
| Ammonium chloride | 0.3 |
| Ammonia (25% strength in water) | ad pH 8.5 |
| Water | ad 100 |

⁷Hydroxyethylcellulose (Hercules)
⁸2-((2-Amino-4-nitrophenyl)amino)ethanol
⁹4-Amino-2-nitrodiphenylamine
¹⁰(3-hydroxypropyl)amino-3-nitrophenol (Clariant)
¹¹C12-C14-fatty alcohol 4.5 EO acetic acid sodium salt (12% active substance in water; INCI name: Sodium Laureth-6 Carboxylate) (KAO)
¹²Cetylstearyl alcohol + 12 EO (INCI name: Ceteareth-12) (COGNIS)

| | |
|---|---|
| 14. Dyeing agent N | |
| Hydrenol ® D | 8.0 |
| C12-C18 fatty alcohol | 2.0 |
| Akypo Soft ® 45 NV | 10.0 |
| Eumulgin ® E1 | 0.5 |
| Eumulgin ® B2 | 0.5 |
| m-Aminophenol | 0.005 |
| 3-Amino-2-methylamino-6- | 0.002 |
| methoxypyridine hydrochloride | |
| 2-Amino-3-hydroxypyridine | 0.24 |
| 2,7-Dihydroxynaphthalene | 0.02 |
| 2-Methylresorcinol | 0.6 |
| p-Tolylenediamine sulfate | 0.73 |
| Resorcinol | 0.13 |
| 2,6-dihydroxy-3,4-dimethylpyridine | 0.1 |
| 2,4,5,6-Tetraaminopyrimidine | 1.3 |
| 5-Amino-2-methylphenol | 0.03 |
| 5-(3-Hydroxyethyl)amino-2- | 0.3 |
| methylphenol | |
| 2-Nitro-4-aminodiphenylamine-2′- | 0.1 |
| carboxylic acid | |
| Ammonium chloride | 0.4 |
| Perfume oil | 0.2 |
| Ammonia (25% strength in water) | ad pH 8.5 |
| Water | ad 100 |
| 15. Dyeing agent O | |
| Hydrenol ® D | 8.0 |
| C12-C18 fatty alcohol | 2.0 |
| Akypo Soft ® 45 NV | 10.0 |
| Eumulgin ® B1 | 0.5 |
| Eumulgin ® B2 | 0.5 |
| 2-Methylresorcinol | 0.48 |
| p-Tolylenediamine sulfate | 0.6 |
| 1-(2′-Hydroxyethyl)-2,5- | 0.6 |
| diaminobenzene | |
| Resorcinol | 0.21 |
| 2,6-dihydroxy-3,4-dimethylpyridine | 0.66 |
| 2,4,5,6-Tetraaminopyrimidine | 3.0 |
| 3-Methyl-4-aminophenol | 0.16 |
| 3-Amino-2-chloro-6-methylphenol | 0.11 |
| 5-Amino-2-methylphenol | 0.03 |
| 1,5-Dihydroxynaphthalene | 0.002 |
| Ammonium chloride | 0.2 |
| Mirapol ® A-15¹³ | 1.5 |
| Perfume oil | 0.2 |
| Histidine | ad pH 8.5 |
| Water | ad 100 |

¹³polymeric quaternary ammonium salt (about 64% active substance; INCI name: Polyquaternium-2) (RHONE-POULENC)

| | |
|---|---|
| 16. Dyeing agent P | |
| Hydrenol ® D | 5.0 |
| C12-C18 fatty alcohol | 2.0 |
| Texapon ® NSO | 3.0 |
| Ammonium sulfate | 0.5 |
| 5-Amino-2-methylphenol | 0.56 |
| 3-Methyl-4-aminophenol | 0.055 |
| p-Tolylenediamine sulfate | 0.65 |
| Resorcinol | 0.14 |
| 4-Aminophenol hydrochloride | 0.6 |
| 4-Amino-2-nitrodiphenylamino-2′- | 0.05 |
| carboxylic acid | |
| 6-Nitro-1,2,3,4-tetrahydroquinox- | 0.1 |
| aline | |
| Ammonia (25% strength in water) | ad pH 8.5 |
| Water | ad 100 |
| 17. Dyeing agent Q | |
| Hydrenol ® D | 5.0 |
| C12-C18 fatty alcohol | 2.0 |
| Texapon ® NSO | 3.0 |
| Ammonium sulfate | 0.5 |
| Bis(2,4-diaminophenoxy)propane | 0.003 |
| tetrahydrochloride | |
| p-Tolylenediamine sulfate | 0.033 |
| Resorcinol | 0.0055 |
| 4-Aminophenol hydrochloride | 0.014 |
| 2-amino-3-hydroxypyridine | 0.0035 |
| Ammonia (25% strength in water) | ad pH 8.5 |
| Water | ad 100 |

For the coloration, 1 part of the above-described dyeing agent was mixed in each case with 0.1 part of an enzyme preparation. The enzyme preparation comprises, based on the amount of protein, 0.3% by weight of enzyme, obtained from Stachybotrys parvispora or Stachybotrys chartarum.

This application preparation was applied to 80% gray mid-blond human hair, left there for 30 minutes and then rinsed out. The results of these colorations, comparable colorations having been obtained with regard to the color direction upon use of the two enzymes mentioned, are given in the table below.

| Dyeing agent | Coloration |
|---|---|
| A | Carrot red |
| B | Birch gray |
| C | Leather brown |
| D | Red brown |
| E | Reddish brown |
| F | Birch gray |
| G | Port wine red |
| H | Agate brown |
| I | Pale orange |
| J | Violet brown |
| K | English red |
| L | Madeira brown |
| M | Gold brown |
| N | Ruby red |
| O | Gamet |
| P | Mahogany |
| Q | Light ash blond |

1. An agent for dyeing keratin fibers comprising, in a cosmetically acceptable carrier, at least one dye precursor, characterized in that it comprises at least one phenol-oxidizing enzyme which can be obtained from Stachybotrys species.

2. The agent as claimed in claim 1, characterized in that the enzyme is purified.

3. The agent as claimed in either of claims 1 or 2, characterized in that the enzyme is chosen from Stachybotrys parvispora, Stachybotrys chartarum, Stachybotrys kampalensis, Stachybotrys theobromae, Stachybotrys bisbyi, Stachybotrys cylindrospora, Stachybotrys dichroa, Stachybotrys oenanthes and Stachybotrys nilagerica.

4. The agent as claimed in any of claims 1 to 3, characterized in that it comprises, as dye precursor, at least one developer component.

5. The agent as claimed in any of claims 1 to 4, characterized in that it comprises, as dye precursor, at least one indole and/or indoline derivative.

6. The agent as claimed in any of claims 1 to 5, characterized in that it comprises at least one coupler component.

7. The agent as claimed in any of claims 1 to 6, characterized in that it further comprises at least one substantive dye.

8. The agent as claimed in any of claims 1 to 7, characterized in that it further comprises at least one surfactant.

9. The agent as claimed in claim 8, characterized in that it comprises a nonionic or an amphoteric surfactant.

10. The agent as claimed in claim 8 or 9, characterized in that it comprises a surfactant combination of at least one anionic surfactant with at least one amphoteric surfactant and/or with at least one nonionic surfactant.

11. The agent as claimed in any of claims 1 to 10, characterized in that it comprises an alkalizing agent.

12. A method of dyeing keratin fibers, characterized in that an agent as claimed in any of claims 1 to 11 is used.

13. The use of a phenol-oxidizing enzyme which can be obtained from Stachybotrys species for the oxidative dyeing of keratin fibers.
